(19) United States
(12) Patent Application Publication
(10) Pub. No.: US 2025/0241851 A1
(43) Pub. Date: Jul. 31, 2025

(54) A SYSTEM AND A METHOD FOR PRODUCING PARTICLES OF ACTIVE PHARMACEUTICAL INGREDIENTS

(71) Applicant: NANOFORM FINLAND OYJ, Helsinki (FI)

(72) Inventors: Kari SEPPÄLÄ, Helsinki (FI); Peetu IHALAINEN, Helsinki (FI); Pia RUNEBERG-ROOS, Helsinki (FI); Maria LUME, Helsinki (FI); Daniel VEIRA CANLE, Helsinki (FI)

(21) Appl. No.: 18/854,409

(22) PCT Filed: Mar. 24, 2023
(86) PCT No.: PCT/FI2023/050169

§ 371 Date: Oct. 4, 2024

(30) Foreign Application Priority Data
Apr. 7, 2022 (FI) 20225299

(51) Int. Cl. A61K 9/00; B05B 7/06

(52) U.S. Cl. CPC A61K 9/0078; B05B 7/06

(57) ABSTRACT

The disclosure relates to a system (100) comprising primary nebulizer (101), a secondary nebulizer (102) and a connecting means (103) therebetween. The secondary nebulizer comprises an atomizer (107) comprising an inlet (109), an outlet (110) and two coaxial passages (111, 112), and a nozzle (108). The passage (111) is connected to the primary nebulizer via the connecting means and serves to carry the aerosol flow comprising API droplets generated by the primary nebulizer to the outlet (110), whereas the passage (112) positions the nozzle. The passages, the nozzle and their respective outlets are arranged relative to one other such that, in use, gas jet introduced through the nozzle shears the droplets of the API at the outlet (110). The invention relates also to a method for producing API particles using the system.

### FIELD

The disclosure relates to systems for producing particles of active pharmaceutical ingredients (APIs) in particular to systems comprising a primary nebulizer for producing an aerosol flow of API droplets, and a secondary nebulizer for subjecting the aerosol to high velocity gas jet. The disclosure also relates to a method for producing particles of API using the system.

### BACKGROUND

Many active pharmaceutical ingredients (APIs) show limited therapeutic efficacy, mainly due to their inadequate dissolution rate to treat the pathology of interest. A large drug particle size creates an additional problem if a specific site of action in the human body must be reached. For this reason, API size reduction using micronization/nanonization techniques is a valid approach to improve the efficacy of APIs.

Various micronization/nanonization techniques have emerged to increase dissolution rates and bioavailability of various APIs. Exemplary techniques include pearl milling, wet milling, spray drying, atomization, sonocrystallization, and supercritical fluid processes.

As defined by WHO an API is “a substance used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings.” Since the APIs form a large and heterogenous population of synthetic and biological drugs, there are no general method for API micronization/nanonization.

Accordingly, there is still need for further systems and methods to reduce particle size of active pharmaceutical ingredients.

### SUMMARY

The present invention is based on the observation that when an aerosol comprising droplets of active pharmaceutical ingredient (API) was subjected to high velocity gas jet, the API particle size was reduced, and particle size uniformity was increased.

Accordingly, it is an object of the present invention to provide a system for producing particles of active pharmaceutical ingredients, the system comprising a primary nebulizer, a secondary nebulizer, and a connecting means therebetween. The secondary nebulizer comprises an atomizer and a nozzle. The atomizer comprises an inlet, an outlet, a first coaxial passage and a second coaxial passage which terminate adjacent to each other at the outlet. The first coaxial passage is in connection with the primary nebulizer via the connecting means and serves to carry aerosol flow comprising API droplets generated by the primary nebulizer, and the second coaxial passage positions the nozzle. The first coaxial passage, the second coaxial passage, the nozzle and their respective outlets being arranged relative to one other such that, in use, gas introduced through the nozzle shears the droplets comprising the API.

It is also an object of the present invention to provide a method for producing particles of pharmaceutical ingredients using the system according to claim 1.

It is still an object of the present invention to provide an atomizer comprising an inlet, an outlet, and a first coaxial passage and a second coaxial passage which terminate adjacent to each other at the outlet, wherein
the second coaxial passage is realized as an elongated cylinder positioned in the middle of the atomizer, and configured to position a nozzle,
the first coaxial passage is realized as an elongated cylinder positioned around the second coaxial passage and arching towards the outlet, the first coaxial passage comprising the inlet.

It is still an object of the present invention to provide a secondary nebulizer comprising the atomizer and a nozzle positioned in the second coaxial passage of the atomizer.

Further aspects of the present technology are described in the accompanying dependent claims.

Exemplifying and non-limiting embodiments of the invention, both as to constructions and to methods of operation, together with additional objects and advantages thereof, are best understood from the following description of specific exemplifying embodiments when read in connection with the accompanying drawings.

The verbs “to comprise” and “to include” are used in this document as open limitations that neither exclude nor require the existence of un-recited features. The features recited in the accompanied depending claims are mutually freely combinable unless otherwise explicitly stated. Furthermore, it is to be understood that the use of “a” or “an”, i.e. a singular form, throughout this document does not exclude a plurality.

### BRIEF DESCRIPTION OF DRAWINGS

FIG. 1 shows a system according to an exemplary non-limiting embodiment of the present invention. The direction of gas and aerosol flow is presented with dotted and solid arrows, respectively.

FIG. 2 shows the cross section of an atomizer according to an exemplary non-limiting embodiment of the present invention.

FIG. 3A shows a top portion of the atomizer of FIG. 2.

FIG. 3B shows the top portion of the atomizer of FIG. 2 and a nozzle positioned in the second passage.

FIG. 4 shows a SEM photograph of HSA particles prepared using a nebulizer of prior art (left) and HSA particles prepared using a system of the present invention wherein the prior art nebulizer was used as a primary nebulizer (right).

FIG. 5 shows a secondary nebulizer according to an exemplary non-limiting embodiment of the present invention.

### DESCRIPTION

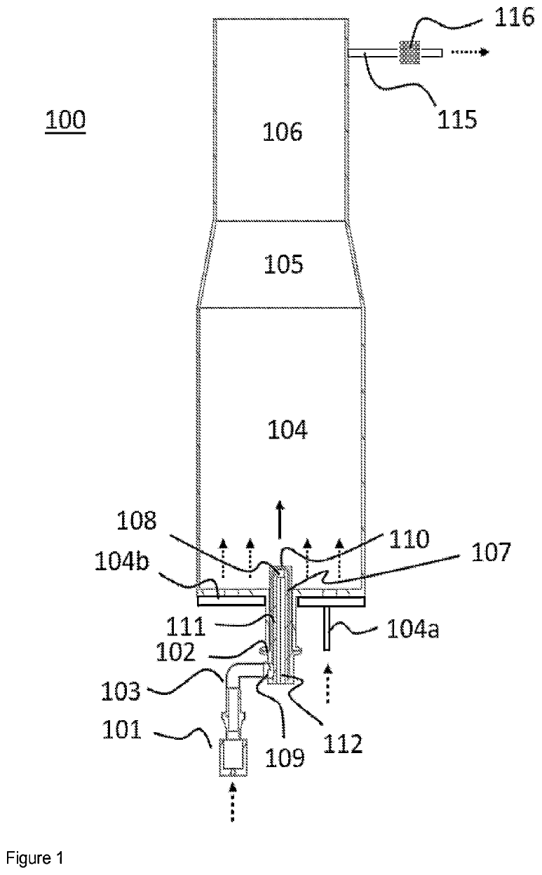

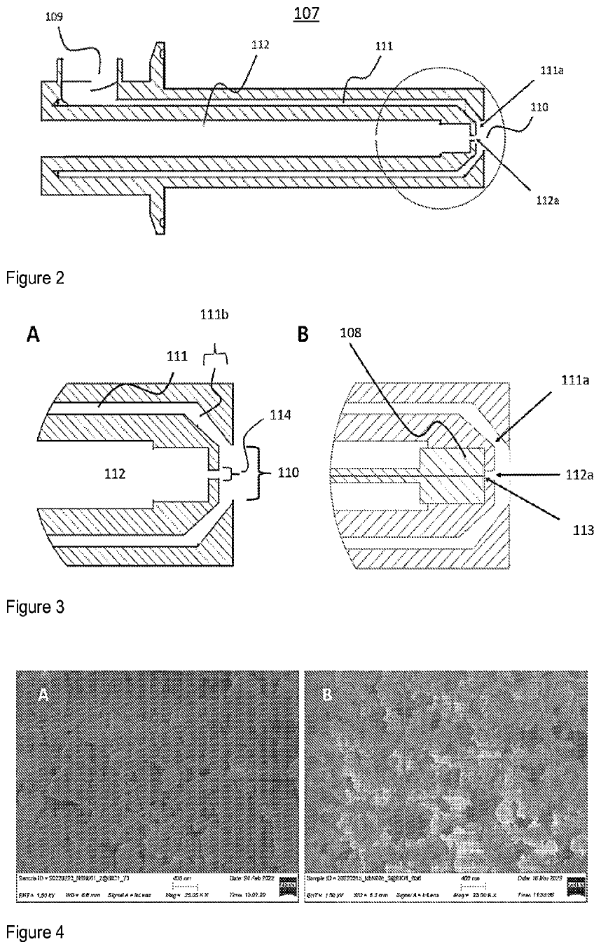

A system 100 for producing particles of organic substances, in particular particles of active pharmaceutical ingredients (API) according to an exemplary non-limiting embodiment is shown in FIG. 1. Details of a secondary nebulizer of the system are best seen in FIGS. 2, 3A, 3B, and 5.

The system 100 comprises a primary nebulizer 101 for producing an aerosol flow comprising droplets comprising particles of organic substances such as particles of API, a secondary nebulizer 102 for exposing the aerosol flow to high velocity gas jet, and a connecting means 103 between the primary nebulizer and the secondary nebulizer. The system comprises also a drying chamber 104, an ionizer 105 and an electrostatic collector 106. The direction of a gas flow in the drying chamber and the aerosol flow is presented in FIG. 1 with dotted arrows and solid arrows, respectively.

The primary nebulizer can be of any type of nebulizer known in the art such as a pneumatic nebulizer, a mechanical nebulizer or an electrical nebulizer. According to an embodiment the primary nebulizer is a pneumatic nebulizer wherein compressed gas such as air is configured to flow at high velocity through a fluid comprising API to turn it into an aerosol which is allowed to flow to the secondary nebulizer via the connecting means.

The secondary nebulizer 102 comprises an atomizer 107, a nozzle 108, an inlet 109, and an outlet 110. The diameter of the outlet 110 is typically 5-20 mm, such as 10 mm.

FIG. 2 shows a cross section of an atomizer 107 of a secondary nebulizer of a system according to an exemplary non-limiting embodiment. The atomizer comprises a first coaxial passage 111 and a second coaxial passage 112 terminating at the outlet 110. The first coaxial passage is in connection to the primary nebulizer via the connecting means. The first coaxial passage serves to carry the aerosol flow generated by the primary nebulizer, and the second coaxial passage is configured to position the nozzle. The design of the second coaxial passage to fit the nozzle is best seen in FIGS. 3A and 3B.

The first coaxial passage 111, the second coaxial passage 112, the nozzle 108 and their respective outlets 111a, 112a, 113 are arranged relative to one other such that, in use, a gas jet introduced through the nozzle towards the outlet 110 shears the droplets comprising the particles of organic substances such as API of the aerosol at the outlet. The system comprises preferably also means for regulating velocity of the aerosol flow and/or the gas flow (not shown).

The second coaxial passage is typically an elongated cylinder positioned in the middle of the atomizer. The outlet of the second coaxial passage is towards the outlet of the atomizer. The first coaxial passage is typically an elongated cylinder positioned around the second coaxial passage, and its head portion 111b arches towards the outlet of the atomizer. Accordingly, the second coaxial passage is realized as an elongated cylinder positioned in the middle of the atomizer and configured to position a nozzle, and the first coaxial passage is realized as an elongated cylinder positioned around the second coaxial passage, the first coaxial passage comprising an inlet 109, and the first coaxial passage arching towards the outlet 110.

Typically, the secondary nebulizer is configured to be in fluid connection with the drying chamber of the system for producing particles of organic substances.

The outlet of the nozzle shown in the figures is an orifice 113 with a diameter typically 0.5 mm or less, such as 0.1-0.5 mm. An exemplary diameter is 0.2 mm. The second coaxial passage comprises preferably a waist 114 at the outlet 112a next to the orifice of the nozzle. An exemplary waist is a cylinder of diameter and length of 1-3 mm. Diameter of the second coaxial passage positioning the nozzle is typically about 10 mm. The diameter of the waist must be larger than the diameter of the orifice of the nozzle in order not to disturb the gas flow.

The drying chamber is configured to dry the aerosol flow comprising the sheared API containing droplets. The drying chamber, in some embodiments, includes a drying chamber air inlet 104a configured to supply air for the drying flow, a fan/pump (not shown) configured to provide a constant pressure to the drying flow, and a heater 104b e.g., a heating element configured to regulate a temperature of the drying flow located at the bottom of the drying chamber when the system is at its operating position.

The ionizer is adapted to expose a particle flow received from the drying chamber. The ionizer produces a flow of negative ions configured to charge the particles to produce a charged particle flow. The system may also comprise a plurality of ionizers.

The electrostatic collector is configured to receive the charged particle flow from the ionizer. According to an exemplary embodiment the electrostatic collector includes a central ground electrode, an external cylinder having a wall with a surface at the predetermined voltage, and an electrostatic collector exhaust 115. The electrostatic collector is configured to deflect the incoming charged particles of the charged particle flow towards the surface of the wall, such that the particles form a layer thereon. Additionally, the system may further include a filter 116 configured to capture dried particles from the electrostatic exhaust that are not collected in the electrostatic collector.

It is also an object of the present invention to provide a method for producing active pharmaceutical ingredients using the system. The method comprises the following steps
a) dissolving or suspending the API to a fluid thereby forming an admixture,
b) atomizing the admixture in the primary nebulizer 101 thereby forming an aerosol comprising droplets comprising the API,
c) allowing the aerosol to flow from the primary nebulizer via the connecting means 103 and the first coaxial passage 111 towards the outlet 110 of the atomizer 107,
d) subjecting the aerosol at the outlet to pressurized gas jet flowing from the nozzle 108 through the orifice 113 towards the outlet, thereby producing an aerosol comprising sheared droplets,
e) drying the sheared droplets thereby producing the API particles, and
f) collecting the API particles.

When the system comprises a drying chamber 104, an ionizator 105, and an electrostatic collector 106, the steps e) and f) comprise
i. exposing the sheared droplets to a drying gas flow to produce a flow of API particles and directing the flow into an area with an ionizer,
ii. exposing the particle flow received from the drying chamber to flow of negative ions to charge the API particles to produce a charged particle flow, and
iii. collecting the charged particles onto an electrostatic collector.

The fluid is any fluid suitable for producing an aerosol comprising droplets of the API. Exemplary fluids are water and ethanol and mixture thereof. The pressurized gas is of any inert gas such as nitrogen, air, and argon or mixture thereof, preferably nitrogen. Pressure of the gas is typically 5-100 bar, such as 5-20 bar. Exemplary APIs suitable for the method are selected from a group consisting of a protein, a nucleic acid, a carbohydrate, a small molecule embedded in a protein, an excipient, and pharmaceutically acceptable salts thereof. The method can also be used for producing particles comprising two or more APIs, excipients, and pharmaceutically acceptable salts thereof.

FIG. 4A shows a SEM photograph of human serum albumin (HSA) particles produced using a commercial nebulizer (Omron C28P), and FIG. 4B shows SEM photograph of HSA particles using a system comprising a secondary nebulizer 102 connected to a commercial nebulizer and a pressure nitrogen flow of 13 bar. As seen from the figures, the use of the system of the present invention reduced the particle size and increased the particle uniformity.

According to another aspect the present invention concerns an atomizer 107 comprising an inlet 109, and outlet 110, and a first coaxial passage 111 and a second coaxial passage 112 which terminate adjacent to each other at the outlet, wherein the second coaxial passage is realized as an elongated cylinder positioned in the middle of the atomizer, and configured to position a nozzle, and the first coaxial passage comprises an inlet 109 and is realized as an elongated cylinder positioned around the second coaxial passage and arching towards the outlet.

Accordingly, the outlet 110, the first coaxial passage 111 and the second coaxial passage 112 terminate adjacent to each other at the outlet 110. The diameter of the outlet 110 is typically 5-20 mm. The second coaxial passage comprises preferably a waist 114 at the outlet. The waist is typically a cylinder in diameter and length of 1-3 mm.

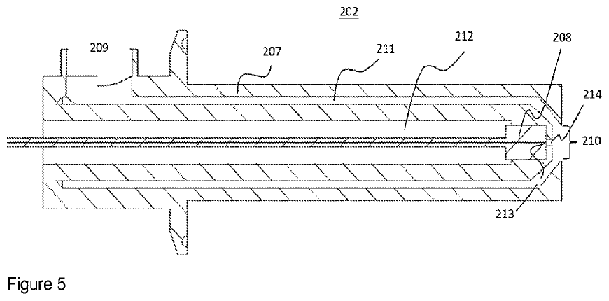

According to a still another aspect the present invention concerns a secondary nebulizer comprising an atomizer and a nozzle. An exemplary secondary nebulizer 202 is shown in FIG. 5. A secondary nebulizer comprises an atomizer 207 comprising an inlet 209, an outlet 210, a first coaxial passage 211, and a second coaxial passage 212. The first coaxial passage and the second coaxial passage configured to terminate adjacent to each other at the outlet, and a nozzle 208, wherein the second coaxial passage is realized as an elongated cylinder positioned in the middle of the atomizer, and positioning the nozzle, and the first coaxial passage is realized as an elongated cylinder positioned around the second coaxial passage and configured to arch towards the outlet, the first passage comprising the inlet. The secondary nebulizer comprises preferably one or more of the following features
The diameter of the outlet is 5-20 mm.
The second coaxial passage comprises a waist 214 at outlet. The waist is a typically a cylinder in diameter and length of 1-3 mm.
The nozzle comprises an outlet 213 realized as an orifice of diameter of 0.5 mm or less, such as 0.1-0.5 mm.
The diameter of the second coaxial passage positioning the nozzle is typically about 10 mm.

1. A system (100) for producing particles of active pharmaceutical ingredients (API), the system comprising a primary nebulizer (101) for producing an aerosol flow comprising droplets comprising the API, a secondary nebulizer (102) for exposing the aerosol flow to gas jet, and a connecting means (103) therebetween, the secondary nebulizer comprising an atomizer (107) and a nozzle (108) wherein
the atomizer comprises an inlet (109), an outlet (110), and a first coaxial passage (111) and a second coaxial passage (112) configured to terminate adjacent to each other at the outlet, the first coaxial passage connected to the primary nebulizer via the connecting means, the first coaxial passage configured to serve to carry the aerosol flow generated by the primary nebulizer, and the second coaxial passage configured to position the nozzle, and
the first coaxial passage, the second coaxial passage, the nozzle and their respective outlets (113, 111a, 112a) being arranged relative to one other such that, in use, gas introduced through the nozzle shears the droplets comprising the API at the outlet (110).

2. The system according to claim 1 wherein
the second coaxial passage is realized as an elongated cylinder positioned in the middle of the atomizer, and configured to position the nozzle, and
the first coaxial passage is realized as an elongated cylinder positioned around the second coaxial passage and arching towards the outlet (110), the first coaxial passage comprising the inlet (109).

3. The system according to claim 1 wherein diameter of the outlet (110) is 5-20 mm, such as 10 mm.

4. The system according to claim 1 wherein outlet (112a) of the second passage comprises a waist (114).

5. The system according to claim 1 wherein the outlet (113) of the nozzle is realized as an orifice of diameter of 0.5 mm or less, such as 0.1-0.5 mm.

6. The system according to claim 1 comprising a drying chamber (104), an electrostatic collector (106) and at least one ionizator (105) therebetween, wherein the drying chamber is connected to the secondary nebulizer.

7. A method for producing particles of active pharmaceutical ingredients (API) by using the system according to claim 1, the method comprising
a) dissolving or suspending API to a fluid to produce an admixture,
b) atomizing the admixture in the primary nebulizer (101) thereby forming an aerosol comprising droplets comprising API,
c) allowing the aerosol to flow from the primary nebulizer via the connecting means (103) and the first coaxial passage (111) to the outlet (110) of the atomizer (107),
d) subjecting the aerosol at the outlet to a pressurized gas jet flowing from the nozzle (108) through the orifice (113) towards the outlet, thereby producing an aerosol comprising sheared droplets,
e) drying the sheared droplets thereby producing the API particles, and
f) collecting the API particles.

8. The method according to claim 7, wherein step e) comprises exposing the sheared droplets to a drying gas flow so as to produce a flow comprising API particles.

9. The method according to claim 8 wherein step f) comprises
i. exposing the flow comprising the API particles to a flow of negative ions to produce a flow comprising charged API particles, and
ii. collecting the charged API particles onto an electrostatic collector.

10. The method according to claim 7 wherein the pressure of the gas is 5-100 bar.

11. The method according to claim 7 wherein the fluid is selected from water and ethanol or mixtures thereof.

12. The method according to claim 7 wherein the gas is nitrogen, argon or mixtures thereof.

13. An atomizer (107) comprising an inlet (109), an outlet (110), and a first coaxial passage (111) and a second coaxial passage (112) configured to terminate adjacent to each other at the outlet, wherein
the second coaxial passage is realized as an elongated cylinder positioned in the middle of the atomizer, and configured to position a nozzle,
the first coaxial passage is realized as an elongated cylinder positioned around the second coaxial passage and configured to arch towards the outlet (110), the first coaxial passage comprising the inlet.

14. The atomizer according to claim 13 wherein diameter of the outlet is 5-20 mm.

15. The atomizer according to claim 13 wherein the second passage comprises a waist (114) at outlet.

16. A secondary nebulizer (202) comprising
an atomizer (207) comprising
an inlet (209),
an outlet (210),
a first coaxial passage (211) and
a second coaxial passage (212), the first coaxial passage and the second coaxial passage configured to terminate adjacent to each other at the outlet, and
a nozzle (208)
wherein the second coaxial passage is realized as an elongated cylinder positioned in the middle of the atomizer, and positioning the nozzle, and the first coaxial passage is realized as an elongated cylinder positioned around the second coaxial passage and configured to arch towards the outlet (210), the first passage comprising the inlet.

17. The secondary nebulizer according to claim 16 wherein diameter of the outlet is 5-20 mm.

18. The secondary nebulizer according to claim 16 wherein the second coaxial passage comprises a waist (214) at outlet.

19. The secondary nebulizer according to claim 16 wherein the nozzle comprises an outlet (213) realized as an orifice of diameter of 0.5 mm or less, such as 0.1-0.5 mm.
